(19) United States
(12) Patent Application Publication
(10) Pub. No.: US 2025/0205267 A1
(43) Pub. Date: Jun. 26, 2025

(54) Dietary Supplement

(71) Applicant: Billy Russell Wall, II, Franklin, TN (US)

(72) Inventor: Billy Russell Wall, II, Franklin, TN (US)

(21) Appl. No.: 18/987,020

(22) Filed: Dec. 19, 2024

Related U.S. Application Data
(60) Provisional application No. 63/613,001, filed on Dec. 20, 2023.

(51) Int. Cl. A61K 31/706; A61K 31/05; A61K 31/122; A61K 31/198; A61K 31/205; A61K 31/352; A61K 31/375; A61K 31/592; A61K 31/593; A61K 31/7084; A61K 31/714; A61K 33/06; A61K 33/30

(52) U.S. Cl. CPC A61K 31/706; A61K 31/05; A61K 31/122; A61K 31/198; A61K 31/205; A61K 31/352; A61K 31/375; A61K 31/592; A61K 31/593; A61K 31/7084; A61K 31/714; A61K 33/06; A61K 33/30

(57) ABSTRACT

The dietary supplement consists of an engineered array of compounds and amalgams that provide balanced and cooperative homeostasis supporting healthy multi-functional aging. The formulary of the supplement array is set and dosed so that each compound and amalgam acts in combination and cooperation to maximize biosynthesis. The various compounds and amalgams of the supplement array act in combination as precursors, cofactors and coenzymes of one another. In an exemplary formulation, the supplement contains nicotinamide riboside, an engineered mitochondrial biogenesis support amalgam, an engineered cell methylation support amalgam, and an engineered sirtuin activation amalgam. The dose and volume of the nicotinamide riboside and the various other amalgams are specifically selected to be efficiently metabolized and absorbed while providing the relevant cooperative biochemical and biological functions to the supplement array.

This application claims the benefit of U.S. Provisional Patent Application, Ser. No. 63/613,001 filed Dec. 20, 2023, the disclosures of both provisional patent applications are hereby incorporated by reference.

This invention relates to a dietary supplement, and in particular, a dietary supplement consisting of an engineered array of compounds and amalgams that provide balanced and cooperative homeostasis supporting healthy multi-functional aging.

### BACKGROUND OF INVENTION

The aging process is believed to relate to the failure of cell components such as the cell nucleus to continue to replicate otherwise healthy cells. This failure is believed to occur as a result of damage which occurs to essential cell components resultant of a decrease in levels of key chemical agents or substances involved in cellular regulation and production. These agents will typically diminish as a result of reduction in efficiency over time of glands and organs in the human body. Reductions in such key chemical agents are believed to cause damage to the RNA and DNA in each cell nucleus which thereby reduces the ability of the cell to reproduce itself which ultimately brings on the aging process which yet further accelerates the decrease in the body's production of the key agents. Therefore, the essential problem in anti-aging management is that of maintaining appropriate levels of key metabolic agents so that the genetic material within each cell will not become damaged or otherwise lose efficiency.

Heretofore a variety of dietary supplements have been developed that exploit singularly or in combination the health and “anti-aging” benefits of various vitamins, nutrients and compounds. The effects and benefits of most common vitamins, nutrients, and compounds are well known in health and biological sciences. However, most of these vitamins, nutrients and compounds are simply taken in the aggregate with little thought to how such vitamins, nutrients and compounds work together or against one another in the body to effect homeostasis.

### SUMMARY OF THE INVENTION

The dietary supplement of this invention consists of an engineered array of compounds and amalgams that provide balanced and cooperative homeostasis supporting healthy multi-functional aging. The formulary of the supplement array is set and dosed so that each compound and amalgam acts in combination and cooperation to maximize bio-synthesis. The various compounds and amalgams of the supplement array act in combination as precursors, cofactors and coenzymes of one another. In an exemplary formulation, the supplement contains nicotinamide riboside, an engineered mitochondrial biogenesis support amalgam, an engineered Cell methylation support amalgam, and an engineered sirtuin activation amalgam. The dose and volume of the nicotinamide riboside and the various other amalgams are specifically selected to be efficiently metabolized and absorbed while providing the relevant cooperative biochemical and biological functions to the supplement array.

Other features and advantages of the dietary supplement of this invention will become more readily apparent to those of ordinary skill in the art by reference to the following detailed description.

### DESCRIPTION OF THE PREFERRED EMBODIMENT

In the following detailed description of the preferred embodiments, reference is made to the accompanying drawings that form a part hereof, and in which is shown by way of illustration specific preferred embodiments in which the invention may be practiced.

These embodiments are described in sufficient detail to enable those skilled in the art to practice the invention, and it is understood that other embodiments may be utilized and that structural and chemical changes may be made without departing from the spirit or scope of the invention. To avoid detail not necessary to enable those skilled in the art to practice the invention, the description may omit certain information known to those skilled in the art. Unless defined otherwise, all technical and scientific terms used herein have the same meaning as commonly understood by one having ordinary skills in the art to which the invention belongs. It should also be understood that various individual components used herein may have more than one type of properties. The following detailed description is, therefore, not to be taken in a limiting sense, and the scope of the present invention is defined only by the appended claims.

The dietary supplement of this invention generally takes the form of one or more tablets or capsules administered orally each day. In certain exemplary cases, the daily dose of the dietary supplement may be formulated as a single tablet or capsule for daily oral administration. In other cases, the dietary supplement may be formulated in multiple tablets or capsules administered orally at set intervals throughout the day.

Each tablet or capsule of the dietary supplement contains a set dosage of an specifically blended and engineered supplement array. The supplement array includes compounds and amalgams that provide balanced and cooperative homeostasis supporting healthy multi-functional aging. The formulary of the supplement array is set and dosed so that each compound and amalgam acts in combination and cooperation to maximize bio-synthesis. Within the supplement array, the various compounds and amalgams act in combination as precursors, cofactors and coenzymes of one another. Each tablet or capsule of the dietary supplement contains a uniform dosage of the supplement array consisting of nicotinamide riboside, an engineered mitochondrial biogenesis support amalgam, an engineered cell methylation support amalgam, and an engineered sirtuin activation amalgam. The dose and volume of the supplement array and the individual compounds and amalgam elements are specifically selected to be efficiently metabolized and absorbed while providing the relevant cooperative biochemical and biological functions to the supplement array.

### Nicotinamide Riboside and Mitoactivation

The supplement array of this dietary supplement includes nicotinamide riboside as a key compound for mitoactivation. Nicotinamide riboside (“NR”) is a pyridine-nucleoside and a derivative of vitamin B3 that acts as a precursor to NAD+ (Nicotinamide Adenine Dinucleotide), a molecule that is critical for proper mitochondrial function, while “Mito Activator” refers to a substance that directly stimulates mitochondrial activity. NR easily converts to a nicotinamide adenine dinucleotide, which is a coenzyme central to metabolism and acts as a mito activator to boost NAD+ levels within the mitochondria, thereby improving the mitochondrial function. Proper mitochondrial function benefits cellular energy production and overall health. When taken as a supplement, NR is converted to NAD+ in the body, leading to an increase in cellular concentration. NR can improve skeletal muscle, brown adipose, and the heart. NR increases NAD content, which improves mitochondrial energy, metabolism, and DNA repair. NR also promotes mitochondrial fusion in the diabetic heart by activating the SIRT 1/PGC 1/DPAR signaling pathway.

### Mitochondrial Biogenesis Support Amalgam

The supplement array further contains an amalgam specifically engineered for mitochondrial biogenesis support. Mitochondrial biogenesis is the process by which cells increase mitochondrial numbers. Mitochondrial biogenesis is activated by numerous different signals during times of cellular stress or in response to environmental stimuli, such as aerobic exercise. The mitochondrial biogenesis support amalgam contains at least 5 mg of nicotinamide adenine dinucleotide hydrogen (“NADH”), at least 50 mg of coenzyme Q10 (“COQ10”), and at least 20 mg of vitamin C.

NADH is a pyridine nucleotide and carries an electron for both anaerobic and aerobic metabolism. NADH is a key signaling molecule for the electron transport chain for generating ATP and driving mitochondrial growth and replication. In cellular metabolism, NAD is involved in redox reactions, carrying electrons from one reaction to another, so it is found in two forms: NAD+ is an oxidizing agent, accepting electrons from other molecules and becoming reduced; with H+, this reaction forms NADH, which can be used as a reducing agent to donate electrons. These electron transfer reactions are the main function of NAD. It is also used in other cellular processes, most notably as a substrate of enzymes in adding or removing chemical groups to or from proteins, in post-translational modifications. Generally, higher NADH levels means more energy production to stimulate mitochondrial biogenesis.

Coenzyme Q10 (“COQ10”) shuttles electrons between complexes I, II, and III. COQ10 in combination with NADH has shown to improve mitochondrial dysfunction and increase oxidative stress which is linked to myalgic encephalomyelitis and chronic fatigue syndrome (“ME/CFS”). COQ10 and NADH together improve overall fatigue perception and cognitive fatigue. COQ10 and NADH are also known to positively effect sleep quality. Vitamin C provides amino acids to help the adrenal glands which under-function in people with ME/CFS.

Mitochondrial biogenesis is the process by which cells increase mitochondrial numbers. Mitochondrial biogenesis is activated by numerous different signals during times of cellular stress or in response to environmental stimuli, such as aerobic exercise. The capacity for mitochondrial biogenesis has been shown to decrease with age, and such decreased mitochondrial function has been associated with diabetes and cardiovascular disease. Aging and disease can induce changes in the expression levels of proteins involved in the fission and fusion mechanisms of mitochondria, thus creating dysfunctional mitochondria.

Cell methylation Support Amalgam

The supplement array further contains an amalgam specifically engineered for Cell methylation support. Cell methylation is a biological process by which methyl groups are added to the DNA molecule. Each of the tablets and capsules of the supplement array includes a Cell methylation support amalgam. The methylation support amalgam contains at least 10 mg betaine, at least 10 mg of vitamin B12 (as Cyanocobalamin), at least 5 mg of dimethylglycine (DMG), at least 5 mg of vitamin D2, at least 5 mg of vitamin D3, at least 5 mg of magnesium (as magnesium oxide), and at least 5 mg of zinc (as zinc citrate).

Each component or element in the amalgam supports and/or assists in the biochemical process which covalently adds methyl groups to a variety of molecular targets, such as DNA, lipids, neurotransmitters, proteins and other vital elements in the body. Vitamin B12 is essential for DNA Methylation. Magnesium increases 5-Methylcytosine DNA Methylation. Zinc is a cofactor for enzymes involved in cell DNA methylation. Vitamin D2 assists in regulating DNA methylation and gene expression. Vitamin D3 also increase DNA Methylation. Dimethylaglycine (DMG) is a non-protein amino acid methyl donor for methylation. Each component is selected and engineered to work in harmony as cofactors with the other compounds and amalgams of the supplement array to promote methylation in the users during double-stranded deoxyribonucleic acid replication.

### Sirtuin Activation Amalgam

The supplement array also contains an amalgam specifically engineered for sirtuin activation. Sirtuins are a family of signaling proteins involved in metabolic regulation. Each of tablets and capsules of the supplement array contains a sirtuin activation amalgam. The sirtuin activation amalgam contains at least 20 mg of resveratrol, and at least 20 mg of fisetin. Both resveratrol and fisetin act as precursors for NADH. Both resveratrol and fisetin are antioxidants and sirtuin-activating compounds that regulate cellular hormesis and that help cells stay healthy. Fisetin acts as a shield for Resveratrol, slowing its breaking down and being metabolized in the liver, thus increasing the amount of resveratrol in the bloodstream.

Resveratrol is in the polyphenol group of stilbenes. Resveratrol has been shown to boost mitochondrial activity by stimulating NAD+ levels to activate sirtuins. Resveratrol binds with sirtuins closing the polypeptide binding pocket. Research and clinical testing has shown resveratrol to effect antioxidants on free-radicals, and have anti-cancer, cardioprotective, anti-inflammatory benefits, as well as improving lung function, glucose homeostasis and systolic blood pressure.

Fisetin is a flavonoid and a senolytic that makes apoptosis accrue in senescent cells. Fisetin has also been shown to be reactive in many different assays of biological activities, raising the possibility that any drug generated from fisetin would have too many side effects to be useful. Consequently, the dose of fisetin ranges between 20 mg and 500 mg to avoid adverse effects in the user. Fisetin activates sirtuins, particularly SIRT 1 and enhances deacetylate activity. Fisetin has been shown to reduce inflammation, neuroprotection and senescent cells to enhance anti-active (anti-cancer) activity.

### Cross Amalgam Combination Benefits

The supplement array of this invention is specifically designed and engineered to work within the body's chemistry. The various compounds and amalgams of the supplement array have uniquely blended and engineered benefits and interactions in cross combinations or combinations of with the compounds of the other amalgam arrays in the supplement. While each individual compound has a known therapeutic effect and benefit, the cross combination of the compounds from different amalgams creates additional unique therapeutic effects and benefits beyond their known and intended use within the specific amalgam.

With regard to sirtuin activation, Nicotinamide Riboside Chloride (NR) activates sirtuins by boosting NAD+ levels as a precursor to NAD+, which is a cofactor for sirtuin enzymes to function properly. Co-Enzyme Q10, found in the mitochondrial biogenesis support amalgam, activates sirtuins, particularly SIRT1 through mitochondrial function. Several compounds from the methylation amalgam have direct effects on sirtuin activation. Vitamin D2 assists auto-deacetylation on Lysosome Biology, (Lys-bio) which activates SIRT 1. Vitamin D3 activates VDR/SIRT 1/SIRT 3, which aids aging muscles. Magnesium activates an anti-aging gene SIRT 1, which has benefits for cardiovascular disease and diabetes. Zinc maintains structural integrity of SIRT Proteins, particularly SIRT 1. Dimethylglycine (DMG) also activates SIRT 1 and the PGC 1 Network.

With regard to cellular methylation, Vitamin C, from the mitochondrial biogenesis support amalgam, promotes DNA demethylation and histone demethylation. Co-enzyme Q10, also from the mitochondrial biogenesis support amalgam, produces S-adenosyl methionine (SAMe) that transfers methyl-groups to DNA. Fisetin, from the sirtuin activation amalgam, regulates cell methylation by inhibiting DNA, DMT 1, which has anti-aging benefits. Resveratrol, also from the sirtuin activation amalgam, reduces PTN promoters in MCF-7 cells and increases methylation in oncogenes and inhibits transcriptional activity in breast cancers.

With regard to mitochondrial biogenesis support, NR increases mitochondrial biogenesis and protects against diet induced obesity and insulin resistance. Fisetin, from the sirtuin activation amalgam, increases mitochondrial biogenesis and neuronal cells and improves mitochondrial dysfunction in hepatocytes. Resveratrol, also from the sirtuin activation amalgam, is an antioxidant and anti-inflammatory and promotes mitochondrial biogenesis in various tissues, skeletal muscle, and blood vessels. Several compounds from the methylation amalgam have direct effects on mitochondrial biogenesis. Vitamin B12 regulates oxidative stress and maintains genomic stability, thereby influencing telomere integrity and cellular aging and mitochondrial biogenesis. Vitamin D2 deficiency leads to mitochondrial dysfunction and impaired biogenesis. Vitamin D2 improves mitochondrial biogenesis and function. Vitamin D3 increases mitochondrial biogenesis markers and will increase protein content in cells and increase total antioxidant capacity (TAC). Zinc is a cofactor for many mitochondrial enzymes and subunits of the electron transport chain. Zinc is required for metalloproteases that cleave proteins during mitochondrial maturation. Magnesium is essential for production of Adenosine Triphosphate (ATP) that power's mitochondrial function. Betaine enhances mitochondrial biogenesis and cellular survival, which enhances mitochondrial respiration and membrane potential. DMG is a metabolite involved in choline oxidation that affects mitochondrial function. DMG stimulates reactive oxygen species (ROS) production in liver mitochondria, improves mitochondria function by neutralizing free radicals, improving redox status, and improves gene and protein expression in mitochondrial function, which activates Nrf2/SIRT and 1/PGC 1 networks.

The supplement array is specifically blended and engineered so that various compounds and amalgam elements act and interact as cofactors, coenzymes, cocatalysts, enzyme helpers, essential factors, and substrates, for each compound and amalgam elements to achieve numerous therapeutic results and benefits. In addition, the supplement array is blended and engineered to avoid adverse interactions with other compound and amalgam elements in the array. In addition, the composition and dosage for each compound and amalgam element are selected to balance and optimize bioavailability across the entire supplement array. As a result, the chemical and biological actions and interactions at the cellular level between the various compounds and amalgam elements within the supplement array enhance the therapeutic benefits of this dietary supplement beyond the simple aggregation of the individual compounds and amalgam within the array.

It should be apparent from the foregoing that an invention having significant advantages has been provided. While the invention is shown in only a few of its forms, it is not just limited but is susceptible to various changes and modifications without departing from the spirit thereof. The embodiment of the present invention herein described and illustrated is not intended to be exhaustive or to limit the invention to the precise form disclosed. It is presented to explain the invention so that others skilled in the art might utilize its teachings. The embodiment of the present invention may be modified within the scope of the following claims.

I claim:

1. A dietary supplement for oral administration to an individual, the supplement comprising:
a supplement array deliverable for daily oral administration in one or more tablets and capsules for daily oral administration, the supplement array includes in combination
a) nicotinamide riboside;
b) a first supplement amalgam that acts as cofactors and coenzymes for the supplement array for methylation support of deoxyribonucleic acid;
c) a second supplement amalgam that acts as a co-factor for the nicotinamide riboside for mitochondrial biogenesis; and
d) a third supplement amalgam that co-enables the nicotinamide riboside and the first supplement amalgam for sirtuin activation.

2. The dietary supplement of claim 1 wherein the first supplement amalgam contains betaine, vitamin B-12, dimethylglycine (DMG), Vitamin D, vitamin D3, magnesium, and zinc.

3. The dietary supplement of claim 1 wherein the second supplement amalgam contains nicotinamide adenine dinucleotide hydrogen, coenzyme Q10 and vitamin C.

4. The dietary supplement of claim 1 wherein the third supplement amalgam contains resveratrol and fisetin.

5. The dietary supplement of claim 1 wherein the nicotinamide riboside in each of the one or more tablets and capsules of the supplement array is at least 150 mg.

6. The dietary supplement of claim 2 wherein the first supplement amalgam in each of the one or more tablets and capsules of the supplement array contains at least 10 mg betaine, at least 10 mg of vitamin B-12, at least 5 mg of dimethylglycine (DMG), at least 5 mg of vitamin D, at least 5 mg of vitamin D3, at least 5 mg of magnesium, and at least 5 mg of zinc.

7. The dietary supplement of claim 3 wherein the second supplement amalgam in each of the one or more tablets and capsules of the supplement array contains at least 5 mg of nicotinamide adenine dinucleotide hydrogen, at least 50 mg of coenzyme Q10, and at least 20 mg of vitamin C.

8. The dietary supplement of claim 3 wherein the third supplement amalgam in each of the one or more tablets and capsules of the supplement array contains at least 20 mg of resveratrol, and at least 20 mg of fisetin.
